(19) United States
(12) Patent Application Publication
(10) Pub. No.: US 2012/0143095 A1
(43) Pub. Date: Jun. 7, 2012

(54) BODY MOVEMENT DETECTION DEVICE AND BODY MOVEMENT DETECTION METHOD

(75) Inventor: Fumihiko NAKAMURA, Kyoto-shi (JP)

(73) Assignee: OMRON HEALTHCARE CO., LTD., Muko-shi (JP)

(21) Appl. No.: 13/352,772

(22) Filed: Jan. 18, 2012

Related U.S. Application Data
(63) Continuation of application No. PCT/JP2010/063469, filed on Aug. 9, 2010.

(30) Foreign Application Priority Data
Sep. 4, 2009 (JP) 2009-204477

(51) Int. Cl. A61B 5/11

(52) U.S. Cl. 600/595

(57) ABSTRACT

A body movement detection device that computes a unit-differentiated body movement amount appropriately in tune with a user's life rhythm and a body movement detection method are provided to enhance a user's level of satisfaction. A pedometer provided with an acceleration detection unit that acquires an acceleration that changes with the body movement of a living body, a calculation unit that computes the number of steps based on an acceleration signal and a display unit that displays the computed number of steps includes a storage unit that stores a change condition in which a demarcation of 1 day is set as the unit for aggregating the number of steps. The change condition is constituted by a condition that is determined based on the acceleration signal and a time signal, and the calculation unit computes the number of steps by day.

### TECHNICAL FIELD

This invention relates to a body movement detection device that is able to, for example, accurately detect body movement of a living body for one day in accordance with a life rhythm of the living body, and a body movement detection method.

### BACKGROUND ART

Heretofore, pedometers that use an acceleration sensor to detect change in acceleration resulting from body movement of a living body, and count the number of steps based on acceleration data detected by this acceleration sensor have been provided as devices for detecting and aggregating the amount of body movement of a living body.

Currently available pedometers accumulate the counted number of steps from 0:00 AM to 11:59 PM using time clocked in the pedometer, and output this accumulated step count as the number of steps for one day. A problem with this accumulation method is that for users who are active over the period from 11:59 PM to 0:00 AM the next morning, the pedometer switches to counting the next day's steps while they are still active, and is not in accord with their life rhythm.

A pedometer that is able to automatically store daily step counts when a time preset by the user arrives has been proposed to deal with such problems (see Patent Literature 1).

### Citation List

### Patent Literature

### Patent Literature I: 2003-099754A

### SUMMARY OF INVENTION

### Technical Problem

However, the above-mentioned pedometer has a problem in that step counting will not be in accord with his or her life rhythm if the user does not set the pedometer correctly, and will not necessarily be able to realize step counting to the user's satisfaction.

This invention, in view of the abovementioned problems, has as an object to provide a body movement detection device that is able to compute a unit-differentiated body movement amount appropriately in tune with a user's life rhythm and a body movement detection method, and to enhance the user's level of satisfaction.

### Solution of Problem

This invention is a body movement detection device including an acceleration sensor for acquiring an acceleration that changes with body movement of a living body, a body movement discrimination unit that discriminates body movement based on acceleration data detected by the acceleration sensor, a storage unit that stores the body movement together with a detection time of the body movement, a body movement amount computation unit that computes the body movement as a unit-differentiated body movement amount per prescribed unit of activity, and an output unit that outputs the computed unit-differentiated body movement amount, the storage unit being configured to store a change condition for changing the unit of activity used in computing the unit-differentiated body movement amount, and the body movement amount computation unit changing the unit of activity depending on the change condition which is determined based on the body movement and the detection time of the body movement, and computing the unit-differentiated body movement amount per changed unit of activity.

The acceleration sensor can be constituted by an appropriate sensor that detects acceleration. This acceleration sensor may be configured to output only acceleration or to also output detection time together with acceleration.

The unit-differentiated body movement amount can be an amount obtained by dividing or aggregating the body movement amount resulting from body movement per predetermined unit of activity. Also, the body movement amount can be a measurement amount related to body movement, such as number of steps, travel distance including walking distance or running distance, burned calorie or amount of activity.

The output unit can be constituted by a device that performs output, such as an LCD or similar display device, a communication device that performs data transmission, or an audio output device that performs audio output.

The change condition can be a condition that can be determined based on acceleration data and time data. Specifically, the change condition can be an appropriate condition such as a date change condition for changing a “date” demarcation as the aggregation unit, a home-to-work commute change condition for changing a “home-to-work commute” demarcation as the aggregation unit, a work-to-home commute change condition for changing a “work-to-home commute” demarcation as the aggregation unit, an after-work exercise change condition for changing a “after-work exercise” demarcation as the aggregation unit, or a plurality of these conditions. The body movement detection device can be constituted by a device that is able to detect body movement such as a pedometer or an activity monitor.

According to this invention, a body movement detection device that can compute a unit-differentiated body movement amount appropriately in tune with a user's life rhythm can be provided, and the user's level of satisfaction can be enhanced.

As a mode of this invention, a configuration can be adopted in which the change condition can be configured to include a condition that a body movement stop period during which the body movement amount resulting from body movement is at or below a prescribed amount is at least a prescribed period.

The prescribed amount can be set to zero indicating no change in acceleration or a prescribed amount that is determined in advance.

According to this mode, unit change can be executed on the basis of activity being stopped for a prescribed period during which the user is considered to be inactive due being asleep or the like, enabling measurement and output of the unit-differentiated body movement amount in units that are in tune with the user's actual life rhythm.

Also, as a mode of this invention, the change condition can be configured to include a condition that the body movement stop period that is at least the prescribed period commences at or after elapse of a predetermined normal active period from a detection start time at which detection of body movement per unit of activity is started.

The normal active period can be set to a predetermined time, such as an estimated period from waking up until going to bed, for example.

According to this mode, it is possible to prevent a unit being changed in an unintended situation, such as the date (unit) being changed in a situation where the user is not considered to be asleep, such as when the user is taking a nap or has forgotten to bring his or her body movement detection device, for example.

Also, as a mode of this invention, a plurality of body movement stop periods that are at least the prescribed period are acquired, and the body movement detection device includes a change condition update unit that updates the change condition to a prescribed time that is included in the plurality of body movement stop periods.

The prescribed time can be set to a time that is commonly included in a plurality of body movement stop periods, such as a sleep median time at which the user should be sleeping according to his or her life rhythm, for example.

According to this mode, the determination of an updated change condition can be simplified, and subsequent processing can be efficiently implemented by reducing the processing load.

Also, as a mode of this invention, the unit of activity can be set to a day, and the body movement detection device can include a forcible date changing unit that performs a date change at a predetermined forcible date change time in a case where there is not a body movement stop period that is at least the prescribed period.

The forcible date change time can be set to a time at which to forcibly execute a date change, such as a predetermined time or a time at which a predetermined condition is met, such as a time at which a prescribed period has elapsed since the user woke up, for example.

According to this mode, the date can be forcibly changed to the next day when an elapsed time arrives, with the unit-differentiated body movement amount as the body movement amount for one day. Accordingly, even in the case where the user stays up all night, for example, the body movement amount for one day can be appropriately computed, without including the data for the next day in the body movement amount for one day.

Also, as a mode of this invention, the storage unit can be configured to store, as a bedtime, a time in proximity to when body movement was last detected before passing of a time corresponding to the change condition, and the output unit can be configured to output the bedtime.

The user is thereby able to check his or her bedtime, and can refer back to the bedtime at a later date. Also, the user is able to confirm the time until which the body movement amount for that day was detected by referring to the bedtime.

Also, as a mode of this invention, the storage unit can be configured to store, as a wake-up time, a time in proximity to when body movement was first detected after passing of a time corresponding to the change condition, and the output unit can be configured to output the wake-up time.

The user is thereby able to check his or her wake-up time, and can refer back to the wake-up time at a later date. Also, the user is able to check the time from which the body movement amount for that day was detected by referring to the wake-up time.

Also, this invention can be constituted as a body movement detection device including an acceleration sensor for acquiring an acceleration that changes with body movement of a living body, a body movement discrimination unit that discriminates body movement based on acceleration data detected by the acceleration sensor, a storage unit that stores the body movement together with a detection time of the body movement, a body movement amount computation unit that computes the body movement as a unit-differentiated body movement amount per prescribed unit of activity, and a display unit that displays the computed unit-differentiated body movement amount, the display unit including a sleep information display portion for displaying at least one of a wake-up time, a bedtime, and a sleep period.

The user is thereby able to cheek sleep information, and can check for changes in his or her life rhythm, such as staying up late at night or over or under sleeping, rather than only finding out the step count, and can put this information to use in maintaining his or her life rhythm.

Also, this invention can be constituted as a body movement detection method including the steps of acquiring an acceleration that changes with body movement of a living body as acceleration data, discriminating body movement with a body movement discrimination unit based on the acceleration data, storing the body movement together with a detection time of the body movement in a storage unit, computing the body movement as a unit-differentiated body movement amount per prescribed unit of activity with a body movement amount computation unit, and outputting the computed unit-differentiated body movement amount with an output unit, a change condition for changing the unit of activity used in computing the unit-differentiated body movement amount being stored in the storage unit, and the unit of activity being changed depending on the change condition which is determined based on the body movement and the detection time of the body movement, and the unit-differentiated body movement amount being computed per changed unit of activity, by the body movement amount computation unit.

According to this invention, a unit-differentiated body movement amount can be computed appropriately in tune with the user's life rhythm, and the user's level of satisfaction can be enhanced.

### Advantageous Effects of Invention

According to this invention, a body movement detection device that is able to compute a unit-differentiated body movement amount appropriately in tune with a user's life rhythm and a body movement detection method can be provided, and a user's level of satisfaction can be enhanced.

### BRIEF DESCRIPTION OF DRAWINGS

FIG. 1 is a block diagram showing a configuration of a pedometer which is one type of body movement detection device.

FIG. 2 is an illustrative diagram illustrating display contents displayed on a display unit.

FIG. 3 is an illustrative diagram of an exemplary analysis of a user's life rhythm.

FIG. 4 is a flowchart showing a date-differentiated step count processing operation.

FIG. 5 is a flowchart representing date change condition determination processing in detail.

FIG. 6 is a flowchart representing forcible date change condition adjustment processing in detail.

FIG. 7 is a flowchart representing date change condition adjustment processing in detail.

### DESCRIPTION OF EMBODIMENTS

An embodiment of this invention is described hereinafter together with the drawings.

### Embodiment

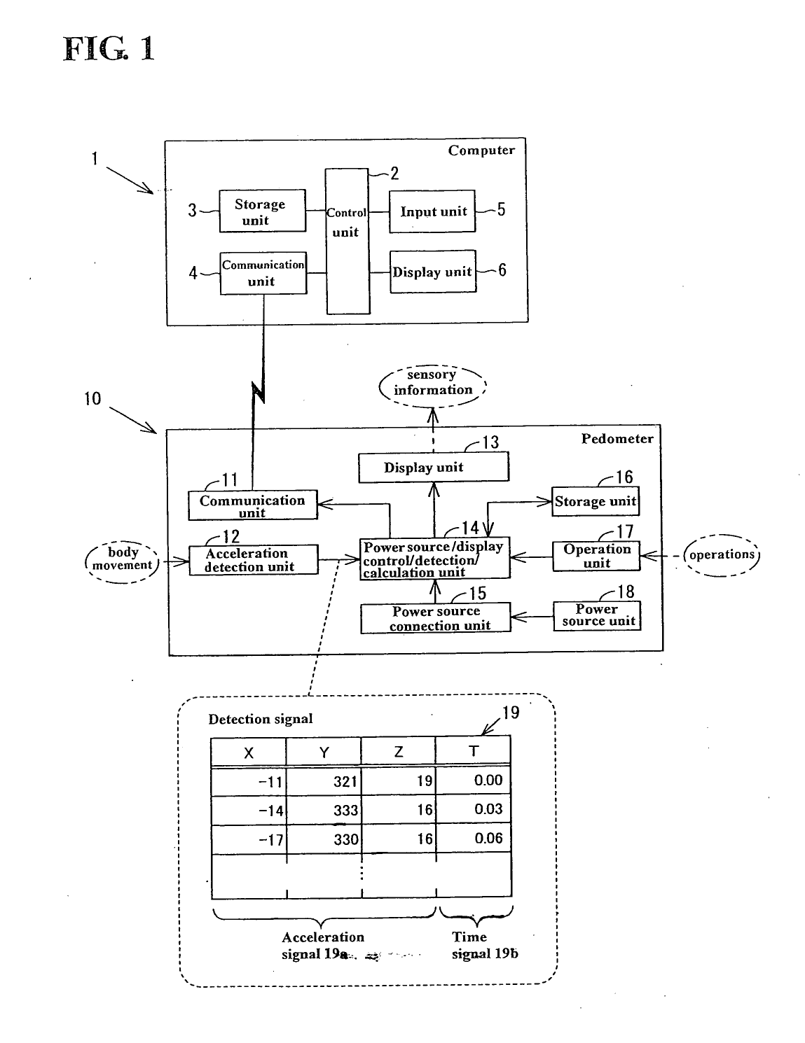

FIG. 1 is a block diagram showing configurations of a pedometer 10 which is a type of body movement detection device and a computer 1 that is capable of communicating with the pedometer 10.

The computer 1 is mainly provided with a control unit 2, a storage unit 3, a communication unit 4, an input unit 5, and a display unit 6.

The control unit 2 is constituted by a CPU, a ROM and a RAM, and executes various control operations and calculations.

The storage unit 3 is constituted by a hard disk or the like, and executes reading/writing of various data in accordance with control signals from the control unit 2.

The communication unit 4 is constituted by a LAN board or the like, and executes data communication with the pedometer 10 in accordance with control signals from the control unit 2.

The input unit 5 is constituted by a keyboard, a mouse or the like, and transmits input signals to the control unit 2 in response to input operations by a user.

The display unit 6 is constituted by an LCD or a CRT, and displays characters and images in accordance with control signals from the control unit 2.

This computer 1 is a personal computer, a handheld terminal or the like, and functions to acquire data such as detection data, step count data and the like from the pedometer 10 and execute appropriate processing such as displaying step counts by date.

The pedometer 10 has a communication unit 11, an acceleration detection unit 12, a display unit 13, a calculation unit 14, a power source connection unit 15, a storage unit 16, an operation unit 17 and a power source unit 18, and is formed to a size that fits in the palm of an average hand so as to be portable.

The communication unit 11 can be constituted by an appropriate communication interface such as a USB (Universal Serial Bus) for cable connection or Bluetooth (registered trademark) for wireless connection. Communication with information processing devices such as personal computers, mobile phones and PDAs (Personal Digital Assistants) that are not shown is thereby realized.

The acceleration detection unit 12 is a sensor that detects the acceleration of oscillations produced by walking, body movement or the like of a user (living body) who is wearing the pedometer 10, and functions to convey a detection signal 19 to the calculation unit 14. The detection signal 19 includes an acceleration signal 19a indicating acceleration and a time signal 19b indicating the time of acceleration detection, with the acceleration signal 19a and the time signal 19b collectively forming a single detection signal 19.

This acceleration detection unit 12 can be constituted by a one-dimensional acceleration sensor that detects acceleration in one direction, a two-dimensional acceleration sensor that detects acceleration in two orthogonal directions, or a three-dimensional acceleration sensor that detects acceleration in three orthogonal directions, with a three-dimensional acceleration sensor that provides a large amount of information being most preferable. In the case of a three-dimensional acceleration sensor, a single detection signal 19 is constituted by four types of data consisting of

X-direction acceleration, Y-direction acceleration, Z-direction acceleration and detection time. Z-direction acceleration is omitted from the four types of data in the case of a two-dimensional acceleration sensor, and Y-direction acceleration and Z-direction acceleration are omitted from the four types of data in the case of a one-dimensional acceleration sensor.

The display unit 13 is constituted by a display device such as an LCD, and displays information in accordance with display control signals from the calculation unit 14. This display information can be appropriate information related to exercise (walking, running, etc.) and life rhythm such as step count, burned calorie, walking distance, date, current time, wake-up time, bedtime, and the like.

The calculation unit 14 is driven by power received from the power source unit 18 via the power source connection unit 15, and functions to receive (detect) the detection signal 19 conveyed from the acceleration detection unit 12 and the operation unit 17, and execute power supply (power source) and operation control (display control) on the display unit 13 and the storage unit 16. Also, the calculation unit 14 executes processing such as calculating the number of steps with reference to walking determination criterion data, one-step determination criterion data or the like stored in the storage unit 16, based on the detection signal 19 conveyed from the acceleration detection unit 12, and aggregation unit adjustment processing for setting data obtained until the date change condition is met as current day data and data obtained after the date change condition is met as next day data.

The storage unit 16 stores data such as date change condition, computed step count, burned calories, walking distance, wake-up time, bedtime and detection data constituting the detection signal 19 detected by the acceleration detection unit 12, and programs for executing processing such as aggregation unit adjustment and step counting.

The operation unit 17 receives appropriate operation inputs, such as operations for inputting user information such as weight and length of stride, date-time input operations for setting the time, display content switching operations for switching the display contents to various types of contents such as step count, burned calories or walking distance, and displayed day switching operations for switching the displayed day to a date such as the current day, one day ago or two days ago, and conveys operation input signals thereof to the calculation unit 14.

The power source unit 18 is constituted by an appropriate power source such as a rechargeable battery or a non-rechargeable battery.

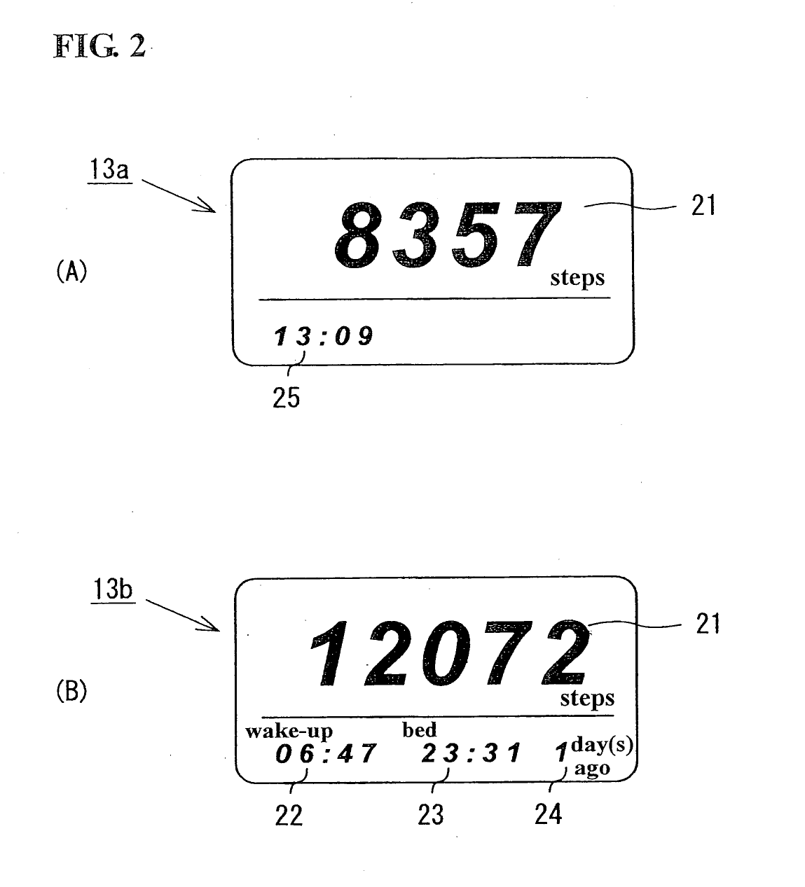

FIG. 2 is an illustrative diagram illustrating display contents displayed on the display unit 13. FIG. 2(A) shows a current day step count display screen 13a displayed on the display unit 13. This current day step count display screen 13a shows a step count 21 and a current time 25.

FIG. 2(B) shows a past step count display screen 13b displayed on the display unit 13. This past step count display screen 13b displays the step count 21, a wake-up time 22, a bedtime 23, and a day number display portion 24.

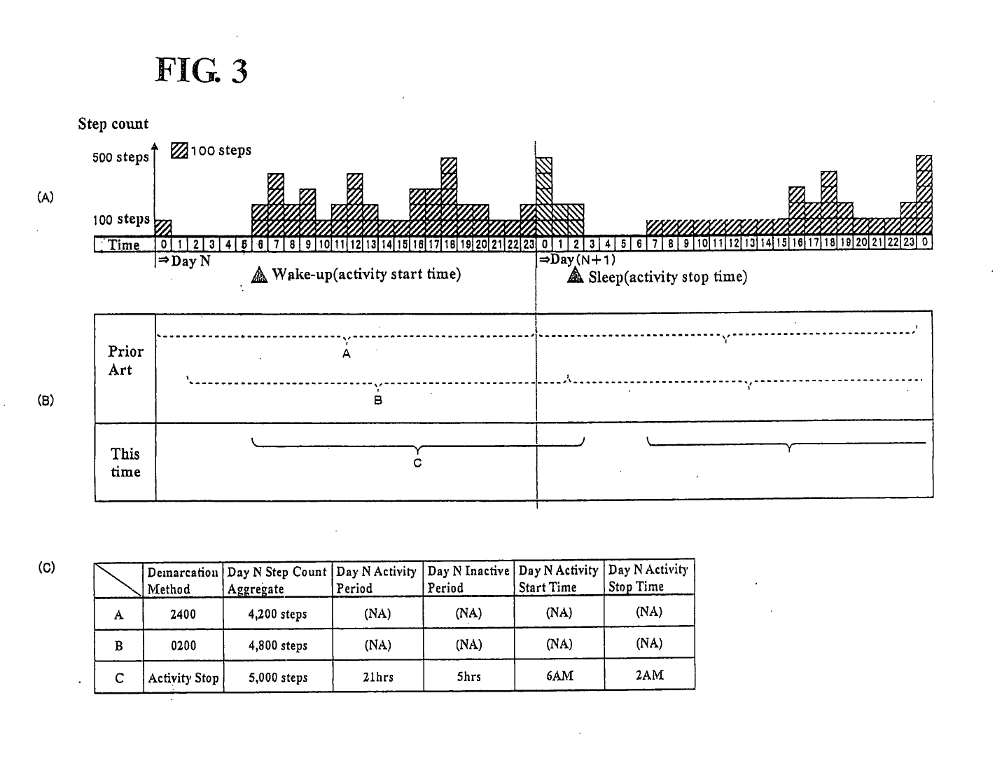

FIG. 3 is an illustrative diagram illustrating an exemplary analysis of a user's life rhythm for appropriately counting the number of steps for one day. FIG. 3(A) shows a time-differentiated step count graph demarcated into units of 1 hour and displaying time-differentiated step counts in time series. In the illustrated example, the user is still awake and active at 12 midnight, with activity being detected for the last time at 2 AM the next day and no activity being detected from then on until 7 AM, so it is clear that the user was asleep during this period.

FIG. 3(B) displays three types of exemplary demarcations A, B and C, in order to illustrate how the aggregate is affected when the demarcation for one day is changed. In the exemplary demarcation A in which one day is marked off at 2400 hrs as was conventionally the case, the step count and the like for the current day are cut off at 12 midnight when the user has not yet gone to bed, and the step count from that point on is included in the next day's step count.

Although the exemplary demarcation B in which one day is marked off at an appropriately set 2 AM sharp is an improvement over the exemplary demarcation A, the step count for the 1 hour from 2 AM is still include in the next day's step count.

In the exemplary demarcation C according to the present embodiment in which one day is marked off after detecting that activity has stopped, one day can be marked off during a period in which the user is considered to be sleeping, and step counting can be executed in tune with the user's life rhythm.

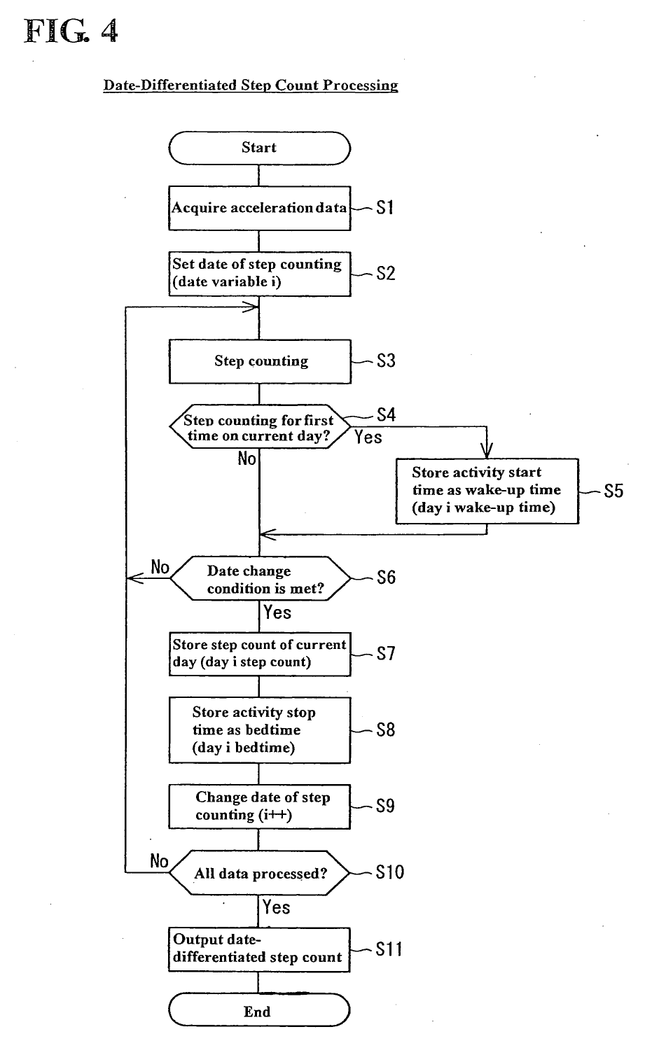

FIG. 4 is a flowchart showing an operation of the calculation unit 14 that executes date-differentiated step count processing. This operation is for computing the number of steps by marking off one day in accordance with the user's life rhythm.

The calculation unit 14 acquires acceleration data (acceleration signal 19a) from the acceleration detection unit 12 (step S1).

The calculation unit 14 sets the date (day i) for step counting (step S2), and performs step counting (step S3). Here, setting the date to be used in step counting can be performed by, for example, setting the current date or by setting a past date for which acceleration data exists.

The calculation unit 14 determines whether step counting is being performed for the first time on the current day (step S4), and, if it is the first time (step S4: Yes), records the operation start time as the wake-up time (day i wake-up time) for the current day (step S5).

If the date change condition is not met (step S6: No), the processing returns to step S3 and step counting is continued.

If the date change condition is met (step S6: Yes), the calculation unit 14 stores the number of steps counted up to that point in the storage unit 16 as the step count (day i step count) for the current day (step S7).

The calculation unit 14 stores the activity stop time, which is the time an acceleration was last detected before the date change condition was met or the time at which accelerations were no longer detected, in the storage unit 16 as the bedtime (day i bedtime) of the current day (step S8).

If, assuming that the date on which step counting is being performed changes and the next day is set as the current day (step S9), all the data has not been processed (step S10: No), the calculation unit 14 returns processing to step S3 and continues the step counting.

If all the data has been processed (step S10: Yes), the calculation unit 14 performs screen output of the step count by date, the wake-up time and the bedtime (step S11), and ends the processing. At this time, transmission output of the step count by date, the wake-up time and the bedtime may be performed from the communication unit 11 to another device such as the computer 1 where necessary. In this case, the other device that receives the step count by date, the wake-up time and the bedtime is able to grasp the user's life rhythm using this information, and put this life rhythm to secondary use.

FIG. S is a flowchart representing in detail the date change condition determination processing for determining whether the date change condition is met at the abovementioned step S6. This operation is for automatically changing the boundary of the date for aggregating the number of steps in accordance with the user's life rhythm.

The calculation unit 14, in the case where it is detected from the acceleration data that activity has stopped (change in acceleration is non-existent or less than or equal to a prescribed value), determines whether activity has stopped for a predetermined estimated sleep period (e.g., 3 hrs) (step S21).

If activity has stopped for the estimated sleep period (step S21: Yes), the calculation unit 14 determines whether the reference activity period (e.g., 14 hrs) has elapsed since the start of activity (day i wake-up time) of the current day (step S22).

If the reference activity period has not elapsed (step S22: No), it is determined that the date change condition is not met (step S25), and the processing is ended. Since the case where this reference activity period has not elapsed arises when the period from the user waking up to going to bed is short, the user has conceivably taken a nap, mislaid his or her pedometer 10, or the like. Thus, a date change is not performed.

If the reference activity period has elapsed (step S22: Yes), the calculation unit 14 determines that the date change condition is met (step S24), and ends the processing. In this case, because a sufficient period has elapsed since the user got up in the morning, and it is moreover determined at step S21 that activity has stopped for long enough for it to be considered that the user is asleep, it is considered to be the timeslot in the user's life rhythm when the user's day has ended and he or she is sleeping.

In the case where, at step S21, activity has not stopped for the estimated sleep period (step S21: No), the calculation unit 14 determines whether the forcible date change condition is met (step S23). This forcible date change condition is for forcibly switching the date to the next day in cases such as where the user stays up all night.

In the case where the forcible date change condition is not met (step S23: No), the calculation unit 14 determines that the date change condition is not met (step S25), and ends the processing.

In the case where the forcible date change condition is met (step S23: Yes), the calculation unit 14 determines that the date change condition is met (step S24), and ends the processing.

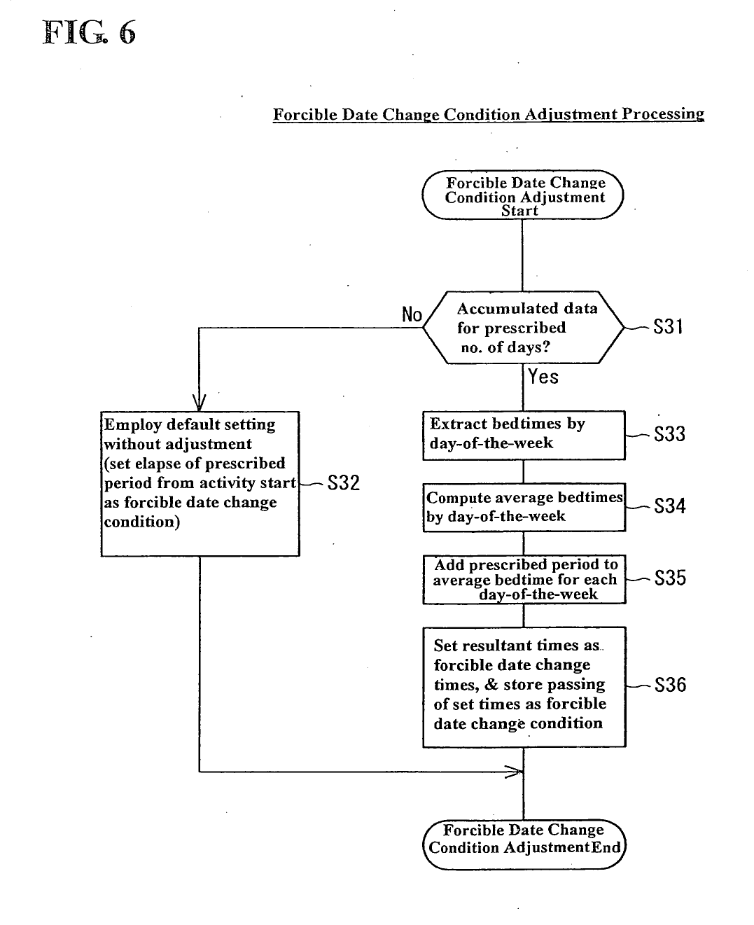

FIG. 6 is a flowchart representing in detail the forcible date change condition adjustment processing for adjusting the forcible date change condition serving as the determination criterion of the abovementioned step S23. This operation is for adjusting the boundary point at which to forcibly change the date in accordance with the user's life rhythm, in order to deal with situations such as the user staying up all night. This forcible date change condition adjustment processing can be executed at a prescribed time (or date-time) such as once a day or once a month.

The calculation unit 14 determines whether data has been accumulated for a prescribed number of days (e.g., 1 month), and, in the case where data has not been accumulated for the prescribed number of days (step S31: No), employs the default setting without adjustment (step S32), and ends the processing. This default setting can be appropriately determined in advance to be, for example, a condition that a prescribed period (e.g., 21 hrs) has elapsed since the user woke up.

In the case where data has been accumulated for the prescribed number of days (step S31: Yes), the calculation unit 14 extracts bedtimes by day of the week (step S33), and computes average bedtimes by day of the week (step S34).

The calculation unit 14 adds a prescribed period (e.g., 3 hrs) to the average bedtime for each day of the week (step S35). This prescribed period preferably is a time period between when the user goes to bed and when the user gets up. This addition processing is not limited to a prescribed period determined in advance, and may be processing for deriving day-of-the-week-differentiated average wake-up times, and deriving an intermediate time between the average bedtimes and the average wake-up times.

The calculation unit 14 sets the times after addition as the forcible date change times and stores the passing of the set times in the storage unit 16 as the forcible date change condition (step S36), before ending the processing.

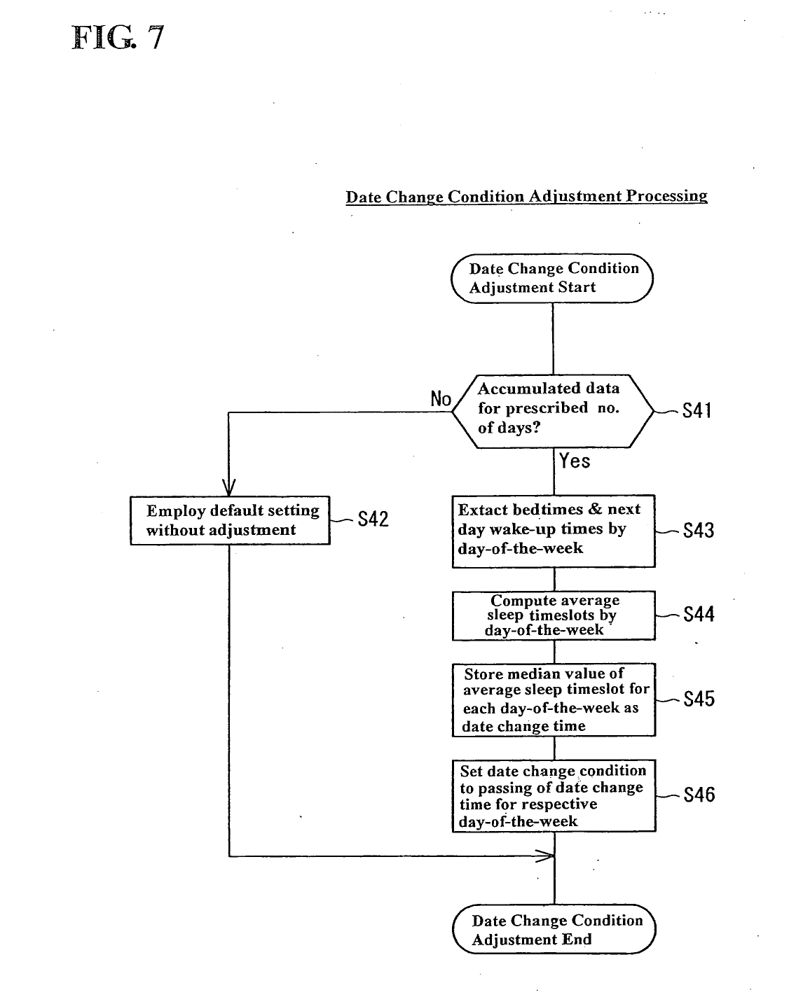

FIG. 7 is a flowchart representing in detail the date change condition adjustment processing for adjusting the date change condition used in the abovementioned determination at step S6. This operation is for reducing the determination processing load by updating the date change condition to a simple determination criterion that is in accordance with the user's life rhythm, if the user's life rhythm can be discriminated. This date change condition adjustment processing can be executed at a prescribed time (or date-time) such as once a day or once a month.

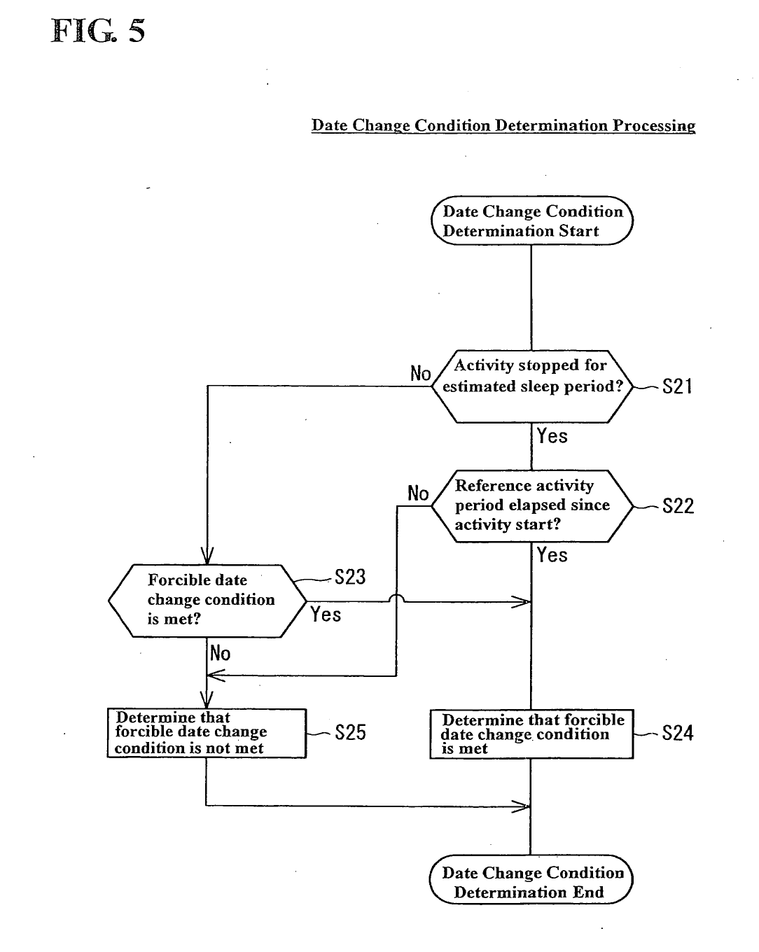

The calculation unit 14 determines whether data has been accumulated for a prescribed number of days (e.g., one month), and, in the case where data has not been accumulated for the prescribed number of days (step S41: No), employs the default setting without adjustment (step S42), and ends the processing. This default setting is the date change condition determination processing described together with FIG. 5.

In the case where data has been accumulated for the prescribed number of days (step S41: Yes), the calculation unit 14 extracts bedtimes and wake-up times by day of the week (step S43), and computes average sleep timeslots (averages of the timeslots from the user going to bed to waking up the next day) by day of the week (step S44).

The calculation unit 14 computes a median value of the average sleep timeslot for each day of the week, and stores these median values in the storage unit 16 as the date change times for each day of the week (step S45).

The calculation unit 14 updates the date change condition by setting the condition as the passing of the date change time for that day of the week (current day of the week) (step S46), and ends the processing.

According to the above configurations and operations, the step count for one day can be computed appropriately in tune with the user's life rhythm, without counting being switched to the next day at a set time regardless of whether the user is awake or asleep as was conventionally the case. In particular, even if there are fluctuations in the user's life rhythm from day to day, due to the user going to bed early, getting up early, sleeping in or staying up late, the date can be changed while the user is asleep in accordance with these fluctuations.

Also, in computing the step count for one day, a date change is executed on the basis of activity being stopped for an estimated sleep period during which the user is considered to be asleep, thus making it is possible to prevent a date change from being performed when activity is stopped for a short period during which the user is not sleeping. Accordingly, the accuracy with which date changes are performed in accordance with the user's life rhythm can be improved.

Also, since a date change is not performed unless a reference activity period has elapsed since the start of activity, it is possible to prevent the date being changed in a situation where the user is not considered to be sleeping, such as when he or she is taking a nap or forgot to bring his or her body movement detection device, for example. Accuracy can thereby be further improved.

Also, since the determination criterion of the date change condition is simplified such that a median value of the average bedtime is set as the date change time in the case where data has been accumulated for a prescribed number of days, step counting in accord with the user's life rhythm can be efficiently performed at a reduced processing load.

Also, since the date is forcibly changed to the next day when the forcible date change time is passed from the start of activity, the body movement amount for one day can be appropriately computed, without including the data for the next day in the body movement amount for one day, even in the case where the user stays up all night, for example.

Also, since the wake-up time 22 and the bedtime 23 are displayed on the past step count display screen 13b of the display unit 13, the user is able to check his or her wake-up time and bedtime, and can refer back to the wake-up time and bedtime at a later date. The user is thereby able to not only find out the step count but also check for changes in his or her life rhythm such as staying up late at night or over or under sleeping, and is able to put this information to use in maintaining his or her life rhythm. Also, the user is able to check the times between which steps for that date were detected by referring to the wake-up time and the bedtime.

In the correspondence between the configuration of this invention and the abovementioned embodiment, a body movement amount computation means of this invention corresponds to the calculation unit 14 that executes step S3 of the embodiment, and hereinafter similarly a storage means corresponds to the storage unit 16, a body movement detection device corresponds to the pedometer 10, an acceleration sensor corresponds to the acceleration detection unit 12, an output means and a display means correspond to the display unit 13, a sleep information display portion corresponds to the wake-up time 22 and the bedtime 23 on the past step count display screen 13b, a forcible date changing means corresponds to the calculation unit 14 that executes steps S23 and S24, acceleration data corresponds to the acceleration signal 19a, time data corresponds to the time signal 19b, a unit-differentiated body movement amount corresponds to the number of steps by date or the number of steps for one day, a body movement amount corresponds to the number of steps, a unit corresponds to a day, a change condition corresponds to the date change condition, a prescribed period corresponds to the estimated sleep period, and a normal active period corresponds to the reference activity period, although this invention is not limited to only the configuration of the abovementioned embodiment, and can obtain many embodiments.

For example, a configuration can be adopted in which time data (time signal 19b) indicating the acceleration detection time is created by the calculation unit 14 adding the time whenever the acceleration signal 19a is received, instead of being output by the acceleration detection unit 12.

Also, the time signal 19b, while being constituted by numerical data that can be synchronized with time and whose numerical value goes up or down every prescribed period, is not limited thereto and may be constituted by time data.

Also, although a configuration was adopted in which the wake-up time and the bedtime are displayed, this invention is not limited thereto, and a configuration can be adopted in which a sleep period from bedtime to wake-up time the next day is displayed.

Also, the processing for counting the number of steps in units of 1 day according to the user's life rhythm is not limited to the pedometer 10, and may be performed by another device such as the computer 1. At this time, the same processing as the flowcharts described together with FIG. 4 to FIG. 7 can be executed by the computer 1. By transmitting detection data (detection signal 19) from the pedometer 10, daily step counts that reflect the user's life rhythm can thereby be computed by the computer 1 that receives this detection data.

As for the data acquisition means capable of acquiring data, the computer 1 is, in this case, not limited to the communication unit 4 that acquires data directly or indirectly from a body movement detection device such as the pedometer 10 or an activity monitor, and may be provided with a connection interface or the like for connecting to the body movement detection device.

Also, in this case, the computer 1 is not limited to the display unit 6, and may be provided with an appropriate output means such as the communication unit 4 or a printing device for outputting the unit-differentiated body movement amount that is computed.

Also, the pedometer 10 may be configured to operate as a stand-alone device, without connecting to the computer 1. In this case, a similar effect to the abovementioned embodiment can also be obtained, and the number of steps can be counted in units of 1 day according to the user's life rhythm.

Also, a configuration may be adopted in which the bedtime and the wake-up time are acquired using a separate sleep measurement device that measures a sleep period, and a date change is performed between the acquired bedtime and wake-up time. In this case, the pedometer 10, in the case where a bedtime and a wake-up time have been acquired by date from a sleep measurement device via the communication unit 11, can perform the date change using the acquired bedtime and wake-up time, and, in the case where a bedtime and a wake-up time have not been acquired from a sleep measurement device via the communication unit 11, can execute the date change condition determination processing of the abovementioned embodiment.

This enables a date change to be performed by the best method according to the environment in which the device is used/installed, since the date change can be performed by the date change condition determination processing if a sleep measurement device is not provided, and the date change can be performed during the acquired sleep period if data can be acquired from a sleep measurement device. Accordingly, the accuracy with which date changes are performed in accordance with the user's life rhythm can be further improved.

Also, a configuration may be adopted in which inputting the wake-up time, the bedtime and the date change time manually with the operation unit 17 is allowed. In this case, the date change condition determination processing of the abovementioned embodiment can be performed under normal conditions, and when a wake-up time, a bedtime and a date change time are input, the input times can be used. Also, in the case where a wake-up time, bedtime and date change time are input, the wake-up time, bedtime and date change time derived by the date change condition determination processing can be updated to the input times.

This enables the user to make alternations by manual input in the case where he or she leads an irregular life that cannot be determined automatically, and to alter the time of the date change to a desired time in the case where a date change is forcibly performed due to the user staying up all night.

Also, a configuration may be adopted in which audio output of the step count, wake-up time, bedtime and the like is performed by an audio output device such as a speaker, instead of or in addition to displaying the step count and the like on the display unit 13. In this case, the user can check the number of steps and the like aurally.

Also, the unit is not limited to date, and a configuration may be adopted in which the number of steps or the amount of activity is aggregated and output for various types of activity such as commuting to and from work and exercise undertaken after work. In this case, the number of steps or the amount of activity can be grasped after being sorted into the various types of activity, and the user's level of satisfaction can be further enhanced.

### INDUSTRIAL APPLICABILITY

This invention is applicable to various devices that detect body movement of a living body, and is particularly applicable to a pedometer or an activity monitor. Also, since the user's life rhythm can be measured, measured data such as wake-up time and bedtime can be output to another device and put to secondary use.

### REFERENCE SIGNS LIST

1 Computer

2 Control unit

3 Storage unit

4 Communication unit

6 Display unit

10 Pedometer

12 Acceleration detection unit

13 Display unit

13b Past step count display screen

14 Calculation unit

16 Storage unit

19a Acceleration signal

19b Time signal

22 Wake-up time

23 Bedtime

1. A body movement detection device comprising:
an acceleration sensor for acquiring an acceleration that changes with body movement of a living body;
a body movement discrimination means for discriminating body movement based on acceleration data detected by the acceleration sensor;
a storage means for storing the body movement together with a detection time of the body movement;
a body movement amount computation means for computing the body movement as a unit-differentiated body movement amount per prescribed unit of activity; and
an output means for outputting the computed unit-differentiated body movement amount,
wherein the storage means is configured to store a change condition for changing the unit of activity used in computing the unit-differentiated body movement amount, and
the body movement amount computation means changes the unit of activity depending on the change condition which is determined based on the body movement and the detection time of the body movement, and computes the unit-differentiated body movement amount per changed unit of activity.

2. The body movement detection device according to claim 1,
wherein the change condition includes a condition that a body movement stop period during which a body movement amount resulting from the body movement is at or below a prescribed amount is at least a prescribed period.

3. The body movement detection device according to claim 2,
wherein the change condition includes a condition that the body movement stop period that is at least the prescribed period commences at or after elapse of a predetermined normal active period from a detection start time at which detection of body movement per unit of activity is started.

4. The body movement detection device according to claim 2,
wherein a plurality of body movement stop periods that are at least the prescribed period are acquired, and
the body movement detection device comprises a change condition update means for updating the change condition to a prescribed time that is included in the plurality of body movement stop periods.

5. The body movement detection device according to claim 2,
wherein the unit of activity is a day, and
the body movement detection device comprises a forcible date changing means for performing a date change at a predetermined forcible date change time in a case where there is not a body movement stop period that is at least the prescribed period.

6. The body movement detection device according to claim 1,
wherein the storage means is configured to store, as a bedtime, a time in proximity to when body movement was last detected before passing of a time corresponding to the change condition, and
the output means is configured to output the bedtime.

7. The body movement detection device according to claim 1,
wherein the storage means is configured to store, as a wake-up time, a time in proximity to when body movement was first detected after passing of a time corresponding to the change condition, and
the output means is configured to output the wake-up time.

8. A body movement detection device comprising:
an acceleration sensor for acquiring an acceleration that changes with body movement of a living body;
a body movement discrimination means for discriminating body movement based on acceleration data detected by the acceleration sensor;
a storage means for storing the body movement together with a detection time of the body movement;
a body movement amount computation means for computing the body movement as a unit-differentiated body movement amount per prescribed unit of activity; and
a display means for displaying the computed unit-differentiated body movement amount,
wherein the display means includes a sleep information display portion for displaying at least one of a wake-up time, a bedtime, and a sleep period.

9. A body movement detection method comprising the steps of:
acquiring an acceleration that changes with body movement of a living body as acceleration data;
discriminating body movement with a body movement discrimination means based on the acceleration data;
storing the body movement together with a detection time of the body movement in a storage means;
computing the body movement as a unit-differentiated body movement amount per prescribed unit of activity with a body movement amount computation means; and
outputting the computed unit-differentiated body movement amount with an output means,
wherein a change condition for changing the unit of activity used in computing the unit-differentiated body movement amount is stored in the storage means, and
the unit of activity is changed depending on the change condition which is determined based on the body movement and the detection time of the body movement, and the unit-differentiated body movement amount is computed per changed unit of activity, by the body movement amount computation means.
